(19) United States
(12) Patent Application Publication
(10) Pub. No.: US 2017/0245656 A1
(43) Pub. Date: Aug. 31, 2017

(54) SLEEP DISORDER TREATMENT DEVICES, SYSTEMS, AND METHODS

(71) Applicant: Hill-Rom Services, Inc., Batesville, IN (US)

(72) Inventors: David L. Ribble, Indianapolis, IN (US); Joshua A. Williams, West Harrison, IN (US); Kirsten M. Emmons, Batesville, IN (US); Brian L. Lawrence, Cincinnati, OH (US); Douglas A. Eckstein, Batesville, IN (US)

(21) Appl. No.: 15/431,985

(22) Filed: Feb. 14, 2017

Related U.S. Application Data
(60) Provisional application No. 62/300,340, filed on Feb. 26, 2016.

(51) Int. Cl. A47C 31/12; A61M 21/02; A47C 27/08

(52) U.S. Cl. CPC A47C 31/123; A47C 27/081; A47C 27/088; A61M 21/02; A61M 2021/0022

(57) ABSTRACT

A sleep system is provided comprising a head support section, a torso support section, a leg support section, and a cradle surface provided on at least an upper portion of the torso support section. The head support section is generally laterally angled at an angle greater than the lateral angle of the torso support section and the leg support section. The cradle surface has at least a partial curvature and a lateral width of from about 12 inches to about 36 inches. The lateral angle of the head support section is at least about 10 degrees, the lateral angle of the torso support section is greater than the lateral angle of the leg support section, and the lateral angle of the leg support section is less than about 10 degrees.

The present application claims the benefit, under 35 U.S.C. §119(e), of U.S. Provisional Application No. 62/300,340, which was filed Feb. 26, 2016 and which is hereby incorporated by reference herein in its entirety.

### BACKGROUND

This disclosure relates generally to systems, methods, and devices for the treatment of sleep disorders. More particularly, but not exclusively, the present disclosure relates to mattresses having features and functions that aid in the treatment of sleep disorders. While various systems have been developed, there is still room for improvement. Thus, a need persists for further contributions in this area of technology.

### SUMMARY

A system, method or apparatus according to this disclosure may comprise one or more of the features recited in the appended claims and/or the following features which, alone or in any combination, may comprise patentable subject matter:

According to one embodiment of the present disclosure, a sleep disorder treatment system is provided comprising a mattress may have a longitudinal length defined by a longitudinal axis of the mattress when the mattress is in its most horizontal position and a lateral width defined by a lateral axis of the mattress when the mattress is in its most horizontal position. The mattress includes a head section having a head support surface to support at least a portion of a person's head. At least a portion of the head support surface is generally sloped in the lateral direction at an angle relative the lateral axis, wherein the angle of the head support surface is from about 10 degrees to about 30 degrees. The mattress also includes a torso section having a torso support surface to support at least a portion of a person's torso, wherein the torso support surface is generally sloped in the lateral direction at an angle relative to the lateral axis, and comprises a cradle surface extending downwardly and laterally from near one side of the torso support section to an opposite side of the torso support section.

According to another embodiment of the present disclosure, a sleep system for supporting a person is provided having a lateral width and longitudinal length and a top surface. The system comprises a first turn bladder, a first flexible pate provided on top of at least a portion of the first turn bladder, a second turn bladder laterally spaced from the first turn bladder. At least a portion of the first flexible plate is positioned on top of the second turn bladder. The system further comprises a second flexible plate positioned on top of at least a portion of the second turn bladder, and an air control system including an air source. The air control system is configured to selectively deliver air to the first turn bladder and the second turn bladder to cause the sleep system to tilt laterally right or laterally left. The flexible plates flex when under top load and the sleep system is laterally tilted.

According to another embodiment of the present disclosure, a sleep system is provided comprising a head support section, a torso support section, a leg support section, and a cradle surface provided on at least an upper portion of the torso support section. The head support section is generally laterally angled at an angle greater than the lateral angle of the torso support section and the leg support section. In some embodiments, the cradle surface has at least a partial curvature and a lateral width of from about 12 inches to about 36 inches. In some embodiments, lateral angle of the head support section is at least about 10 degrees.

Additional features and embodiments, that alone or in combination with any other features of any other embodiments, including those listed above and those listed in the claims, and those described in detail below, may comprise patentable subject matter. Other features and embodiments will become apparent to those skilled in the art upon consideration of the following detailed description of illustrative embodiments. Any feature or aspect disclosed herein, or any embodiment disclosed herein, can be combined with any other feature or aspect or embodiment disclosed herein. One or more features of any embodiment disclosed herein can be combined with one or more features of any other embodiment disclosed herein, and other features can be removed or added to create still further embodiments. Accordingly, many other features, aspects, and embodiments are possible without departing from the spirit, scope, and principles of the present disclosure.

### BRIEF DESCRIPTION OF THE DRAWINGS

Referring now to the illustrative examples in the drawings, wherein like numerals represent the same or similar elements throughout:

FIG. 1 is a top, foot end, perspective view of a mattress according to one embodiment of the present disclosure;

FIG. 2 is a top plan view of the mattress of FIG. 1;

FIG. 3 is a back plan view of the mattress of FIG. 1;

FIG. 4 is a first side elevation view of the mattress of FIG. 1;

FIG. 5 is a second side elevation view of the mattress of FIG. 1;

FIG. 6 is a head end elevation view of the mattress of FIG. 1;

FIG. 7 is a foot end elevation view of the mattress of FIG. 1;

FIG. 8 is a schematic cross-sectional head end view of a mattress according to another embodiment of the present disclosure, having a Z-shaped bladder system, and showing the patient tilted toward their left side;

FIG. 9 is a schematic cross-sectional head end view of the mattress embodiment of FIG. 8, showing the patient tilted toward their right side;

FIG. 10 is a schematic cross-sectional head end view of a mattress according to another embodiment of the present disclosure, having a single turn bladder system, and showing the patient tilted toward their left side; and

FIG. 11 is a perspective view of a Z shaped plate that may be used with some embodiments of the present disclosure.

### DETAILED DESCRIPTION

While the present disclosure can take many different forms, for the purpose of promoting an understanding of the principles of the disclosure, reference will now be made to the embodiments illustrated in the drawings, and specific language will be used to describe the same. No limitation of the scope of the disclosure is thereby intended. Various alterations, further modifications of the described embodiments, and any further applications of the principles of the disclosure, as described herein, are contemplated.

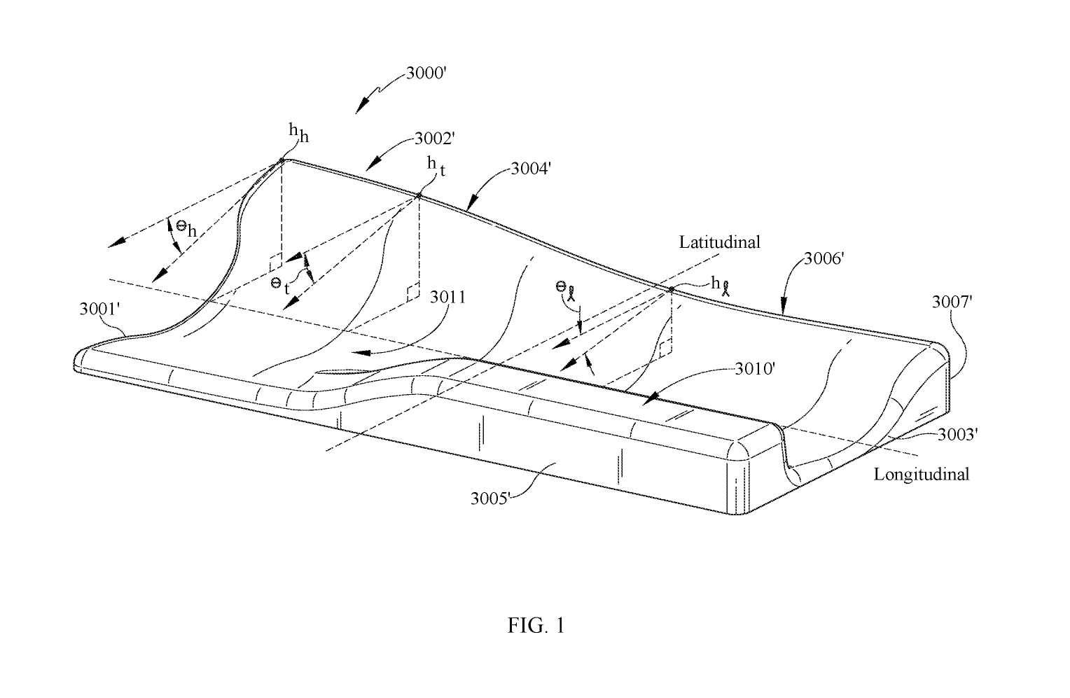

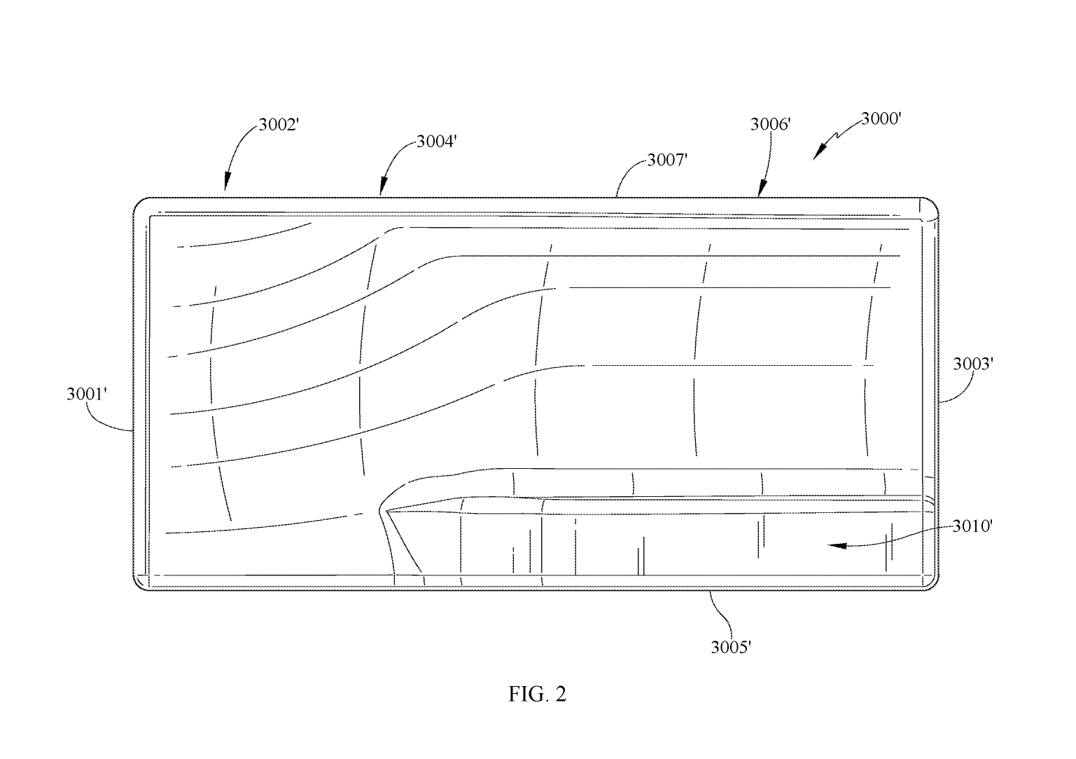

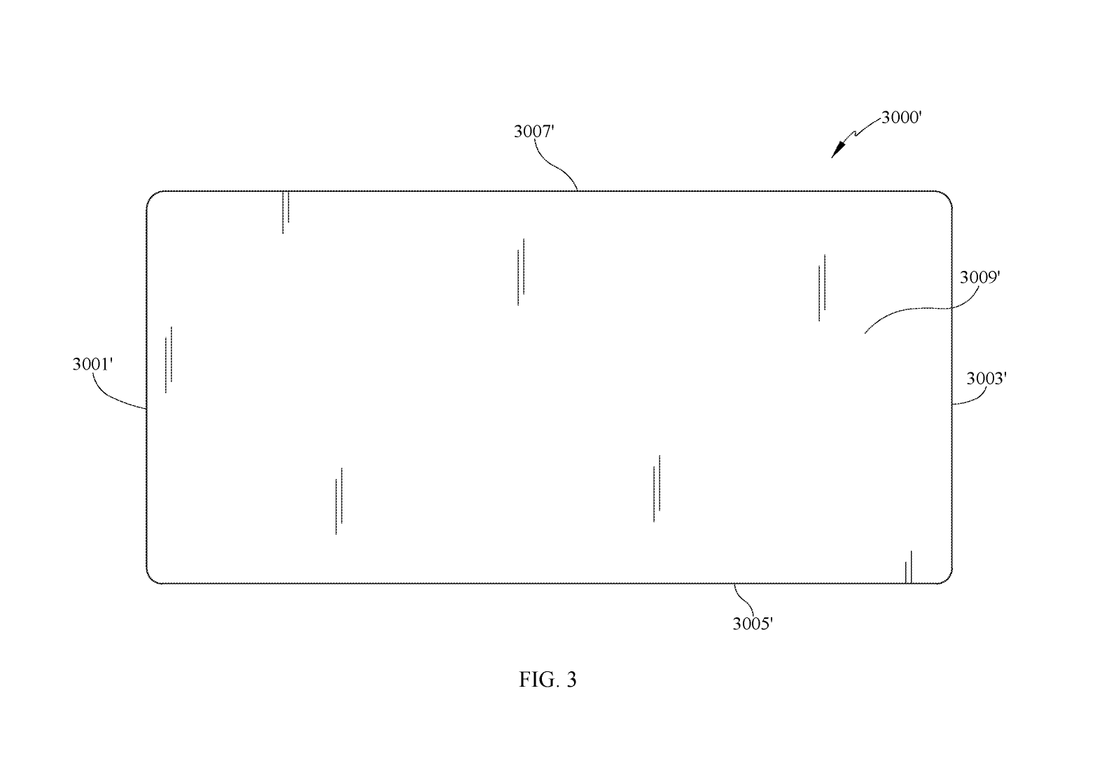

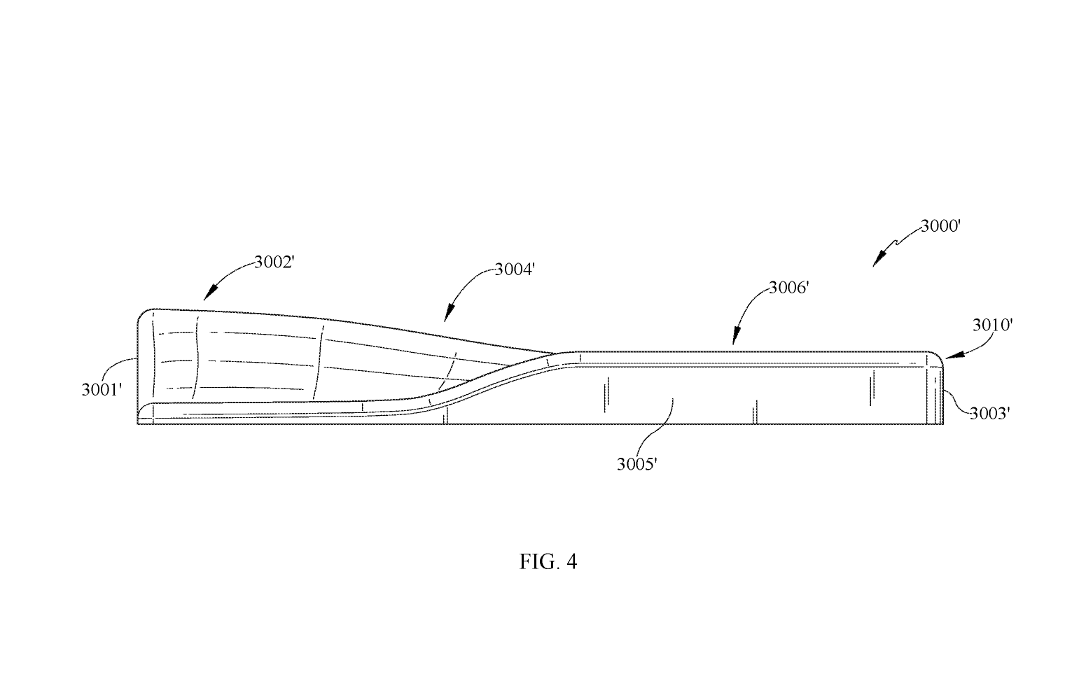

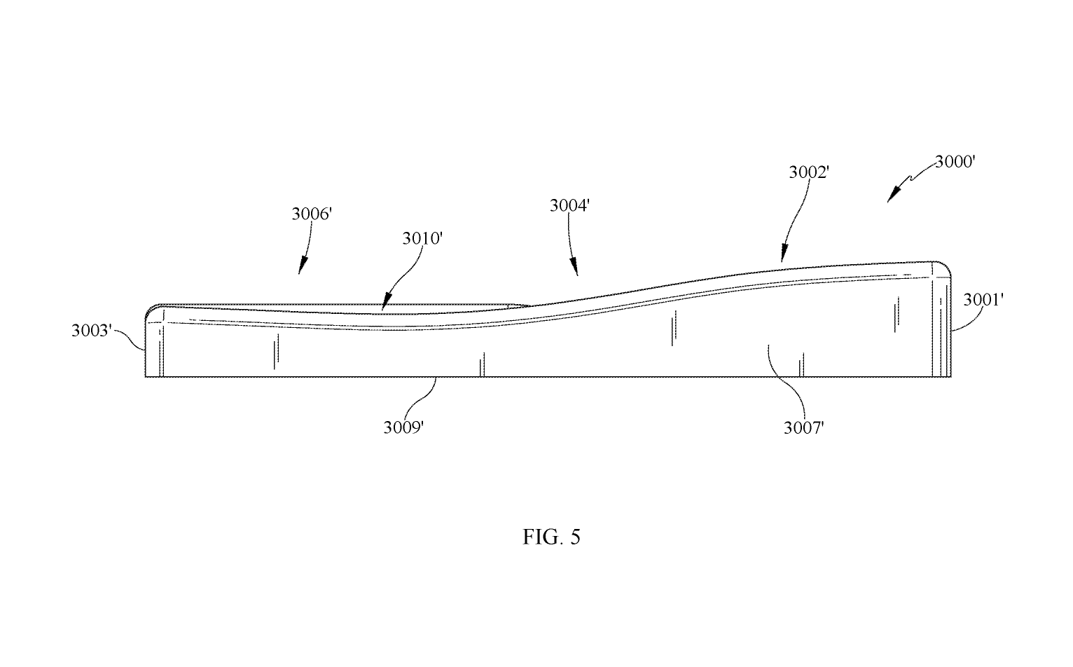

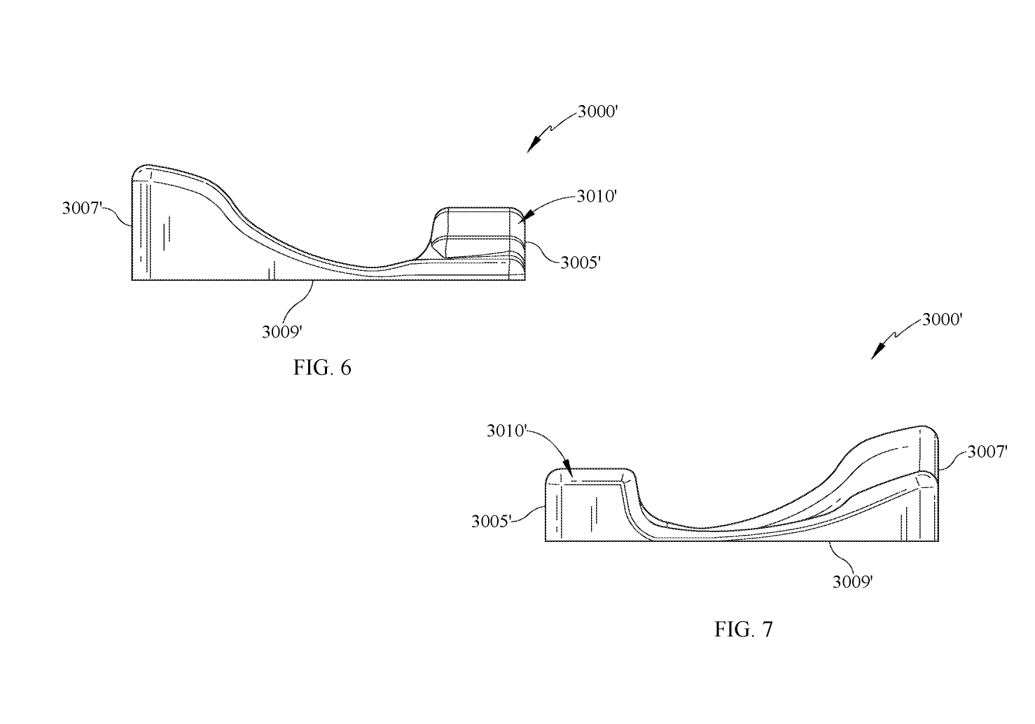

FIGS. 1-7 depict an illustrative embodiment of the present disclosure. In this embodiment, the sleep apparatus is in the form of a mattress 3000′ having a head support section 3002′, a torso support section 3004′, and a leg support section 3006′. Additionally, the mattress 3000′ of this embodiment includes a bolster 3010′. The head support section 3002′, torso support section 3004′, leg support section 3006′, and bolster 3010′ are all made of one or more layers of foam in this embodiment. Other, or additional, fabric materials can be utilized however, and the top layer of the mattress 3000′ can comprise a viscoelastic foam, a cover fabric, fire barrier, casing, and/or ticking material. The mattress 3000′ includes a head end 3001′, a foot end 3003′, a right side 3005′, a left side 3007′, a longitudinal central axis and a lateral (or latitudinal) central axis. In this embodiment, the vertical support sections of the mattress 3000′ is generally slope downwardly in both the lateral direction and in the longitudinal direction. In this example, the sloping occurs in a curved manner in both directions, rather than in a linear or stepped manner (although other embodiments may include other sloping, such as linear, curved, and/or stepped sloping for example).

Each of the sloped lying (person support) sections 3002′, 3004′, and 3006′ include a maximum height. In this example, the head section has a maximum height hₕ, the torso section has a maximum height hₜ, and the leg section has a maximum height h₁, and hₕ is greater than hₜ which is greater than h₁. In some embodiments, there may be irregularly shaped portions of these sections 3002′, 3004′, and 3006′, but the average heights of these sections (taken for example by averaging the maximum heights of all lateral cross sections of each section) are generally different from one another. For example, in one embodiment, the average height of the head section is at least about 20% larger than the average height of the torso section, and the average height of the torso section is at least about 20% larger than the average height of the leg section. Accordingly, the vertical support sections of the mattress 3000′ slope generally downwardly in the longitudinal direction from the head end 3001′ to the foot end 3003′.

Additionally, in this embodiment, the person support sections of the mattress 3000′ slope generally downwardly in the lateral direction from the left side 3007′ toward the right side 3005′. Accordingly, the maximum slope of each of the sections (defined by the tangents to the curves at the highest points hₜ, hₕ, and h₁) relative to horizontal, is indicated by the angles ⊕ₕ, ⊕ₜ, and ⊕₁. In this embodiment ⊕ₕ is greater than ⊕ₜ which in turn is greater than ⊕₁. In other words, the lateral slope of the head section 3002′ is generally greater than the lateral slope of the torso section 3004′, and the lateral slope of the torso section 3004′ is generally greater than the lateral slope of the leg section 3006′. These angles ⊕ₕ, ⊕ₜ, and ⊕₁ of slope could, alternatively, be determined by determining the average slope of lateral cross sections of each section 3002, 3004, and 3006′.

In this embodiment, angle ⊕ₕ, is about 25 degrees, ⊕ₜ, is about 17.5 degrees, and ⊕₁ is about 10 degrees. In some embodiments, the angle ⊕ₕ is from about 10 to about 30 degrees, and the angle ⊕ₜ is from about 0 to about 25 degrees (such as from about 1 to about 20 degrees). In some embodiments, angle ⊕ₕ is at least about 20 degrees, such as from about 20 to about 25 degrees, and the angle ⊕ₜ is at least about 10 degrees, such as from about 10 to about 25 degrees.

Furthermore, in some embodiments, the angle ⊕ₜ is from about 5 to about 15 degrees less than the angle ⊕ₕ. In some embodiments, the angle ⊕ₜ is from about 5 to about 10 degrees less than the angle ⊕ₕ, and in some embodiments the angle ⊕ₜ is about 7.5 degrees less than the angle ⊕ₕ. In some embodiments, the angle ⊕ₜ is from about 15 to about 17.5 degrees.

Moreover, in some embodiments, the angle ⊕₁ is from about 0 degrees to about 15 degrees. In some embodiments, the angle ⊕₁ is from about 0 degrees to about 12.5 degrees, and in some embodiments is about 10 degrees. In some embodiments, the angle ⊕₁ is from about 0 to about 15 degrees less than the angle ⊕ₜ. In some embodiments, the angle ⊕₁ is from about 5 to about 10 degrees less than the angle ⊕ₜ, and in some embodiments the angle ⊕₁ is about 7.5 degrees less than the angle ⊕ₜ.

As shown in FIGS. 1-7, the sleep apparatus in this embodiment includes a cradle surface 3011 that extends laterally across the mattress 3000′ in the areas of the head support section 3002′ and torso support section 3004′. In this embodiment, the cradle surface comprises a generally concave open portion that extends generally downwardly into the mattress material 3000′. The concave portion can generally mimic the lateral curvature of the mid portion of the human back. In some embodiments, the cradle surface extends laterally for at least about 12 inches, such as from about 12 inches to about 36 inches. In some embodiments, the radius of curvature of the curve is from about 5 inches to about 100 inches, such as from about 7 inches to about 50 inches for example, or about 10 to about 40 inches, for example.

In use, the mattress 3000′ can be placed on a bed frame, floor, existing bed or mattress, cot, platform, or other support. The user lies on the top surfaces with the head section surface 3002′ generally supporting the user's head, the torso section surface 3004′ generally supporting the user's torso, and the leg section surface 3006′ generally supporting the user's legs. Due to the angle eh of the head support section 3002′, the side of the user's head is urged to lie at a significant angle (e.g., plus or minus about 35 degrees or more relative to vertical). In this embodiment, in use, a straight line along the approximate surface of the user's face from the ear toward the eye will often lie at an angle (or the plane defined generally by the centerline of the nose), will be at an angle or offset from the vertical up direction and vertical plane. In some embodiments, regardless if the patient is being supported on the patient's back, front, or side, and regardless of whether the patient is sleeping with the face pointed “uphill” relative to the top surface of the mattress 3000′ or pointed “downhill” relative to the top surface, the face is urged to a left or right angle and away from looking straight up to the ceiling, when the mattress 3000′ is in the generally horizontal position. In some embodiments, this urging is to an angle that is 35 degrees or more left or right of the plane that the nose centerline would point if the patient had the nose/face/eyes square with the ceiling, pointing straight ahead. The face can be urged to an angle similar to the lateral plane angle of the top surface of mattress 3000′ (or at least to an angle perpendicular thereto), and certain sleep disorders, such as sleep apnea, may be reduced. In some embodiments, the patient is urged by the surfaces to sleep in a position wherein the soft tissues (e.g., the soft pallet, tongue, uvula, tonsils, pharynx, and/or adenoids) in the upper respiratory tract are at a significant angle relative to the vertical down direction, and/or are less restrictive of the breathing passages so as to minimize apnea events. In some embodiments, because the patient's head twists due to the angled surfaces, torsion is created in the muscles and/or tissues in the neck. This may increase the rigidity of the airway and thereby prevent closing of the upper respiratory tract, reducing possibility of apnea. In this embodiment of FIG. 1, because the mattress has an angled top surface where the angle gradually reduces angle from head end to foot end, it has been found that tolerability/comfort of the mattress 3000′ for sleeping can be improved. Because the mattress 3000′ is made of a flexible material (which is a foam material in this embodiment) when placed on a support deck or frame with a pivoting or raisable leg and/or thigh portion, the leg section 3006′ can be pivoted relative to the torso section 3004′, to thereby create a raised knee and/or thigh, and/or a knee gatch or knee bed in the patient. In some embodiments, this configuration can help resist the migration of the patient toward the foot end and maintain the head in an angled position similar. Furthermore, in this embodiment, the height hₕ of the head support surface 3002′ is generally greater than the longitudinally corresponding height hₜ of the torso support surface 3004′, which is generally greater than the longitudinally corresponding height h₁ of the leg support surface 3006′, so as to create a longitudinal slope on the mattress as well, and causing the body to slope slightly longitudinally downwardly during sleep as well.

The cradle surface 3011 assists in resisting movement of the patient from the desired position on the mattress 3000′ to thereby help maintain the patient's head in the desired position for as long as possible during sleep, and/or to increase perceived comfort and stability of the mattress 3000′. The bolster 3010′ of this embodiment likewise assists in resisting movement of the patient from the desired position. In this embodiment, the cradle surface 3011 is formed by forming the top surface of the foam mattress 3000′ in the desired shape. However, other embodiments are possible, such as by using side members, or by using solid interior plates, as will be described in more detail below with respect to some embodiments. Additionally, the top surface of the mattress 3000′, and the other mattresses described herein, can be covered with a thin layer of quilting or a fabric material, and/or with a visco-elastic foam material, for additional comfort (and/or to assist in maintaining the person in the desired sleeping position for as long as possible during sleep.)

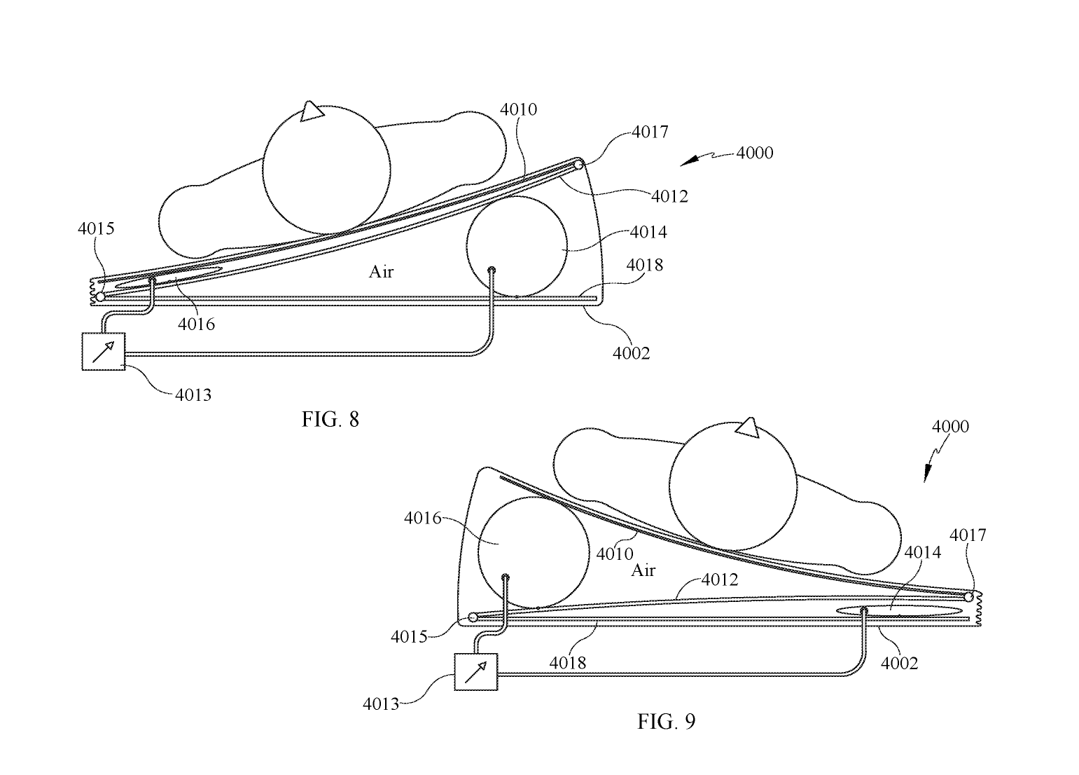

FIG. 8 is a schematic cross-sectional head end view of a mattress 4000 according to another embodiment of the present disclosure, showing the patient tilted toward the left. FIG. 9 is a schematic cross-sectional head end view of the mattress 4000 of FIG. 8, showing the patient tilted toward the right. In this embodiment, the mattress 4000 includes a pair of bladders 4014 and 4016 spaced laterally from each other. The left side bladder 4016 is positioned below an upper plate 4010 and above an intermediate plate 4012. The right side bladder 4014 is positioned below the intermediate plate 4012 but above a lower plate 4018. The plates 4010 and 4012 are connected on the right side by a hinge 4017 so as to pivot relative to one another on one side of the mattress, while the plates 4012 and 4018 are connected on the left side via hinge 4015, so as to pivot relative to one another on the right side of the mattress. These plates 4010, 4012, and 4018 form a generally Z shaped configuration (with a bladder in between each adjacent pair of plates.)

Each bladder 4016 and 4014 can be separately inflated via air control system 4013, which includes a valving system. The valving system can be opened at to allow for air to flow from the air source into the bladder 4016, but not to bladder 4014, or can be opened to allow for air to flow into the bladder 4014, but not bladder 4016. Accordingly, the left and right sides of the mattress 4000 can be selectively inflated, to cause the mattress to turn left or right, to one lateral side or the other, causing the patient to turn one way or the other.

In this embodiment, the plates 4010, 4012, and 4018 are flexible in nature, so as to provide some give under the weight of the patient from the top. Accordingly, as shown in FIGS. 8 and 9, the top surface 4000 of the mattress forms a cradle surface during the turn operations, so as to help maintain the patient at the desired location on the mattress.

The mattress components of this embodiment are enclosed in a ticking material 4002, which can comprise a fabric material. Foam may also be included as desired, to allow for increased comfort and/or support at desired locations. For example, the bladders 4016 and 4014 can be as long as approximately one third of the mattress (along the head end and part of the torso end), while the remainder of the mattress (remainder of torso section and leg section) can transition to horizontal, such as via an angle foam piece. As another example, the lower ˜⅔rd of the mattress 4000 can be shaped like the lower ⅔rd of the mattress shown in FIG. 1, and made from foam, as that embodiment is. Thus, in the embodiment of FIGS. 8 and 9, the mattress 4000 would transition from a large lateral angle near the head section to a small (or zero) lateral angle near the leg section.

In some embodiments the bladder 4014 or 4016 can be inflated to cause the head support surface to have a lateral angle of at least about 20 degrees, and the lateral angle is less in the torso support surface (e.g., at least about 10 degrees, but less than about 20 degrees), due to a smaller volume of the bladder near the torso section, or due to a transition to foam material having a smaller angle. To measure these angles, because the surface is non-linear, a straight linear path can be made from the uppermost point at the start of the inclined support surface to the lowermost point at the bottom of the inclined support surface, and that line compared to horizontal. (The same method can be utilized for measuring the other angles described herein.)

The plate 4010 of FIGS. 8 and 9 causing the cradle surface is larger than the patient width in this example, and can be from about 12 inches to about 36 inches, such as from about 15 to about 25 inches, for example, depending on what size patient the mattress is being designed or configured for. The curvature created by the flexing of the plate 4010 can be from about 5 to about 100 inches in radius, for example, such as about 7-50 inches, or about 10 to about 40 inches, or about 28.5 inches.

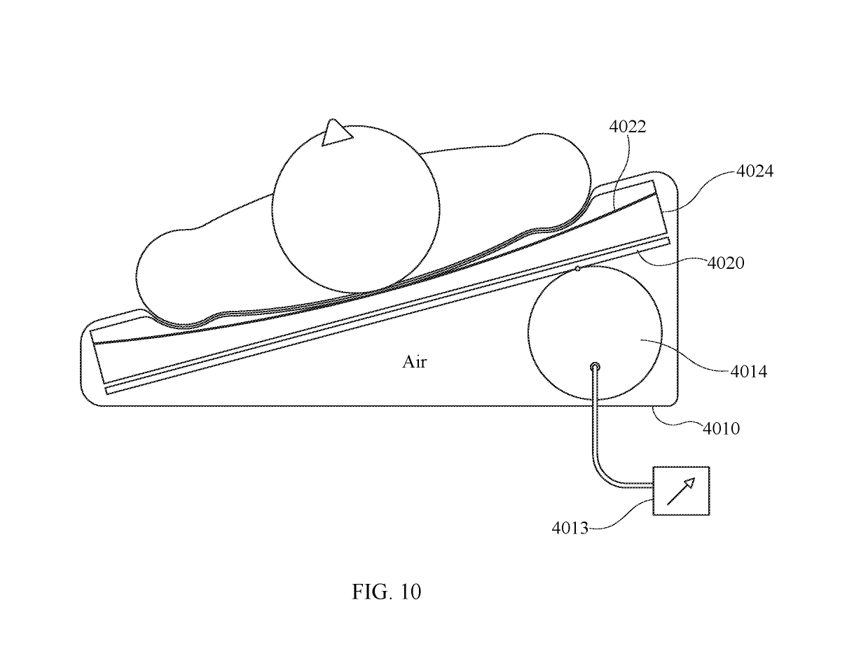

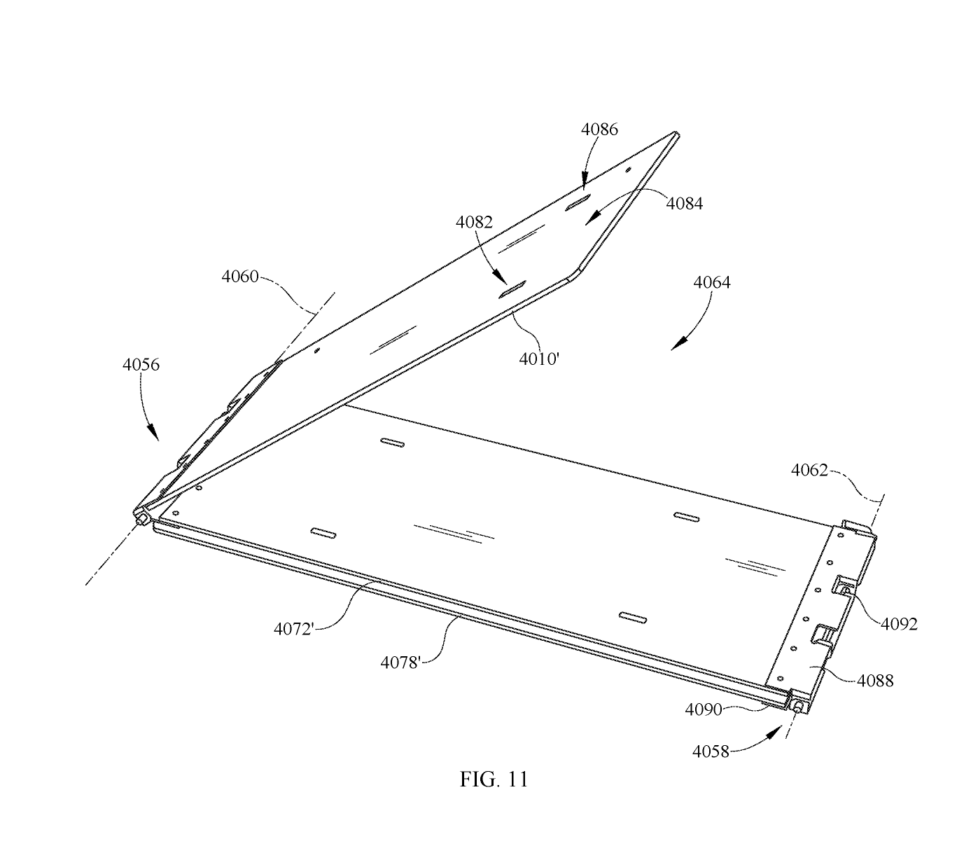

FIG. 10 is a schematic cross-sectional head end view of a mattress according to another embodiment of the present disclosure, having a single turn bladder system, and showing the patient tilted toward their left side. Here, the system includes only a single bladder (bladder 4014) which is inflated by the air control system 4013 at desired times. If desired, a right turn bladder can be placed under the plate 4020 on the opposite side of the bladder 4014, in order to achieve turn in the other direction. As described above, the bladders can be placed only in the head and torso sections, but not in the leg section, so as to achieve a rotation in the head and torso area, but not the foot area (which may be supported by flat foam, non-tilted air bladders, or another support surface.)

In the embodiment of FIG. 10, a rigid plate 4020 is tilted upwardly and toward the left when the bladder 4014 is inflated. Above the plate 4020 is a topper system comprising a rigid, curved plate 4022 embedded in foam 4024. The curved plate 4022 has a radius of 28.5 inches in this example. In some embodiments, the radius of curvature of the plate is from about 5 inches to about 100 inches, such as from about 7 inches to about 50 inches for example, or about 10 to about 40 inches, for example. From the edge of the plate to its lowest point, a drop of 0.25 inches to 4 inches may be made, such as 1 inch for example.

In other embodiments, the curvature of the plate 4022 is noncontinuous. For example, the radius, or amount, of curvature could be greater on one lateral side of the plate than on the other. For instance, the curvature could be greater on the left (downhill) side of the plate 4022 shown in FIG. 10 than on the right (uphill side) of the plate, such that the plate forms a tilted generally J shape. This can provide a greater cradling effect on the downhill side than on the uphill side. Other shapes are possible as well, such as C shapes, and other continuous or non-continuous curved shapes.

As shown in FIG. 10, the mattress of this embodiment allows the patient to immerse into the top of the surface. In some embodiments, the amount of immersion in the mattress is between about 0.25 inches to about 4 inches (if air bladder 4014 is deflated and the mattress is horizontal), such as 1 inch for example, due to the foam 4024. The amount of immersion may be more on the left (downhill) side of the patient, than on the right (uphill side) of the patient, in this embodiment. Other ways of achieving cradling of the tilted patient via cradling are also possible. For example, in the embodiment of FIGS. 8 and 9, some immersion can be achieved through the air bladders 4014 and 4016 and the flexibility of the plates 4018, 4010, and 4012. Returning again to FIG. 10, the mattress is enclosed, and the components held together, by a cover, fabric, or ticking 4010.

FIG. 11 is a perspective view of a flexible Z shaped plate that may be used with some embodiments of the present disclosure. In this embodiment, the assembly causing the lateral turn includes a hinged support plate assembly 4064 which has two hinges 4056 and 4058 that define respective pivot axes 4060 and 4062. The hinges 4056 and 58 are positioned on opposite sides of the hinged support plate assembly 4064 so that the pivot axes 4060 and 4062 lie parallel to the longitudinal length of the mattress on opposite sides.

A pair of inflatable bladders can be positioned between the upper plate 4070 and intermediate plate 4072 of the hinged support plate assembly 4064 and a second pair of bladders 4074 and 4076 can be positioned between the intermediate plate 4072 and a lower plate 4078 as shown in FIG. 11. It should be understood that the plates 4070, 4072, and 4078 are flexible structures constructed of a resin composite, such as ABS plastic, but sufficiently stiff to hold the load between the interface between the bladders and the plates over the entire plate structure. For example, the plates 4070, 4072, and 4078 may have a flexural modulus in the range of 1 to about 10 GPa such as 2.5 GPa for example, and a strength of 10 to 70 MPa. In some embodiments, a polymer that is 1/16 to about 1 inch thick, such as ¼ inch thick ABS for example, can provide suitable flexibility. Other thermoplastics could be utilized as well. The upper plate 4070 (and plate 4010 of FIG. 8) may flex between about 1 and about 10 inches (from its end to the side of the patient, to the middle directly under the patient), with support provided from the bladders and/or foam beneath the plate.

Each bladder can be secured to an adjacent plate 4070, 4072, or 4078 by a respective strap that is secured to the bladder and extends through an opening at one end of the respective plate 4070, 4072, or 4078 and lies on the side of the respective plate 4070, 4072, or 4078 opposite the bladder for a length and is then extends through another opening to reengage the bladder. The interaction of the strap 4080, the bladder, and the respective plate secures the bladder relative to the plate. The engagement of the strap with the plate can maintain the position of the bladder relative to the plate.

The hinges 4056 and 4058 include brackets secured to the plates that are engaged by a rod. For example, as shown in FIG. 11, hinge 4058 is formed by a bracket 4088 which is secured to intermediate plate 4072 and a bracket 4090 which is secured to lower plate 4078. The brackets 4088 and 4090 engage so that several in each bracket 4088 and 4090 align along the pivot axis 4062 so that a rod 4092 can be slid along the pivot axis 4062 to secure the bracket 4088 and 4090. The brackets 4088 and 4090 are movable relative to one another by pivoting on the rod 4092 relative to one another to change an angle between the intermediate plate 4072 and the lower plate 4078.

The various aspects of the above referenced embodiments can be applied to any of a variety of other embodiments. For example, any of the above aspects may be applied, in combination, or individually, to any of the embodiments shown or described in Appendix A of U.S. Provisional Patent Application No. 62/300,340 which is already incorporated by reference herein, and/or to any of the embodiments shown or described in US Patent Application Publication No. 2015/0335507, the entire disclosure of which is hereby incorporated herein by reference. For example, the cradling, concavity, and/or flexibility aspects and components described above may be applied, in combination, or individually, to any of the embodiments shown in FIGS. 1-146 of the Appendix A drawings, or described in the Appendix A text. As another example, the components described in U.S. Patent Application Ser. No. 62/168,596, the entire disclosure of which is hereby incorporated herein by reference, may be utilized to create the desired turn for the embodiments described herein.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

What is claimed is:

1. A sleep disorder treatment system, comprising:
a mattress may have a longitudinal length defined by a longitudinal axis of the mattress when the mattress is in its most horizontal position and a lateral width defined by a lateral axis of the mattress when the mattress is in its most horizontal position, wherein the mattress comprises:
a head section having a head support surface to support at least a portion of a person's head, wherein at least a portion of the head support surface is generally sloped in the lateral direction at an angle relative the lateral axis, wherein the angle of the head support surface is from about 10 degrees to about 30 degrees; and
a torso section having a torso support surface to support at least a portion of a person's torso, wherein the torso support surface is generally sloped in the lateral direction at an angle relative to the lateral axis, and wherein the torso support section comprises a cradle surface extending downwardly and laterally from near one side of the torso support section to an opposite side of the torso support section.

2. The system as recited in claim 1, wherein the cradle surface is concave, wherein the cradle surface extends laterally a distance of from about 12 inches to about 36 inches, and wherein the cradle surface comprises at least one curved surface having a radius of curvature from about 5 inches to about 100 inches.

3. The system as recited in claim 1, wherein the angle of slope of the head support surface is at least about 20 degrees, and the angle of slope of the torso support surface is at least about 10 degrees.

4. The system as recited in claim 1, wherein the cradle surface is created by flexing of the torso support section during use.

5. The system as recited in claim 1, further comprising at least one turn bladder configured to adjust the slope the torso support section.

6. The system as recited in claim 1, wherein the cradle surface comprises a rigid concave backing.

7. The system as recited in claim 6, wherein the cradle backing is made of polycarbonate.

8. The system as recited in claim 6, wherein the cradle backing has a radius of from about 7 inches to about 50 inches.

9. The system as recited in claim 6, wherein the cradle surface comprises a layer of cushioning material covering the rigid concave cradle backing.

10. The system as recited in claim 1, further comprising a bolster, wherein the bolster extends along at least a portion of the torso support surface.

11. A sleep system for supporting a person having a lateral width and longitudinal length and a top surface, comprising a first turn bladder;
a first flexible plate provided on top of at least a portion of the first turn bladder;
a second turn bladder laterally spaced from the first turn bladder, wherein at least a portion of the first flexible plate is positioned on top of the second turn bladder;
a second flexible plate positioned on top of at least a portion of the second turn bladder;
an air control system including an air source, wherein the air control system is configured to selectively deliver air to the first turn bladder and the second turn bladder to cause the sleep system to tilt laterally right or laterally left, wherein the flexible plates flex when under top load and the sleep system is laterally tilted.

12. The sleep system as recited in claim 11, wherein the top surface creates a cradle surface when the flexible plates flex.

13. The sleep system as recited in claim 11, wherein the sleep system comprises a head support surface having an angle of at least about 20 degrees, and a torso support surface having an angle of at least about 10 degrees.

14. The sleep system as recited in claim 11, wherein the sleep system comprises a head support surface having a first lateral angle, a torso support surface having a second lateral angle, and a leg support surface having a third lateral angle, wherein the first lateral angle is greater than the second lateral angle, and wherein the second lateral angle is greater than the third lateral angle.

15. A sleep system, comprising
a head support section;
a torso support section;
a leg support section, wherein the head support section is generally laterally angled at an angle greater than the lateral angle of the torso support section and the leg support section;
a cradle surface provided on at least an upper portion of the torso support section.

16. The system as recited in claim 15, wherein the cradle surface is generally concave and has a lateral width of from about 12 inches to about 36 inches.

17. The system as recited in claim 15, wherein the cradle surface comprises a rigid, generally curved plate, and a cushioning material provided on top of the plate.

18. The system as recited in claim 17, wherein the rigid plate comprises a polycarbonate material and has a radius of curvature of from about 5 to about 100 inches.

19. The system as recited in claim 15, wherein the cradle surface comprises a flexible plate comprising a thermoplastic having a thickness of between about ⅛ inch to about ½ inch.

20. The system as recited in claim 15, wherein the cradle surface comprises at least two flexible plates and at least two turn bladders, each bladder being provided underneath at least one plate.
